(19) United States
(12) Patent Application Publication
(10) Pub. No.: US 2021/0179300 A1
(43) Pub. Date: Jun. 17, 2021

(54) MICRO-AUGER POWDER FILLING APPARATUS

(71) Applicants: Baxter International Inc., Deerfield, IL (US); Baxter Healthcare SA, Glattpark (Opfikon) (CH)

(72) Inventors: Zhiwei CHOU, San Jose, CA (US); Dean GARCIA, Franklin, WI (US)

(21) Appl. No.: 16/769,840

(22) PCT Filed: Dec. 6, 2018
(86) PCT No.: PCT/US2018/064255

§ 371 Date: Jun. 4, 2020

Related U.S. Application Data
(60) Provisional application No. 62/596,407, filed on Dec. 8, 2017.

(51) Int. Cl. B65B 1/12; A61M 5/31

(52) U.S. Cl. CPC B65B 1/12; A61M 2202/064; A61M 5/3129

(57) ABSTRACT

A syringe filling system includes a syringe, a powder source, a funnel, a rotatable auger, and a motor. The syringe has a dispensing end and a barrel chamber. The funnel is configured to hold powder from the powder source. The rotatable auger is positioned within the funnel and is configured to transport powder from the funnel through the dispensing end of the syringe to fill the barrel chamber of the syringe with a predetermined quantity of powder. Additionally, the motor is configured to rotate the auger to cause powder to be transported from the funnel to the syringe.

### CROSS-REFERENCE TO RELATED APPLICATIONS

This application claims priority to U.S. Provisional Patent Application No. 62/596,407 filed Dec. 8, 2017, entitled “MICRO-AUGER POWDER FILLING APPARATUS,” which is incorporated herein by reference in its entirety.

### BACKGROUND

Syringes are filled with various fluids or powders. For example, powder syringes, such as hemostatic syringes dispense fluids or powders, such as wound sealant (e.g., hemostatic powder) through their dispensing ends. The dispensing ends may be luer tips that interface with other components such as fluid lines and containers. Syringes are typically filled from the plunger end using various techniques including vacuum filling, powder feeding, etc.

An improved syringe filling system and method is needed accordingly.

### SUMMARY

The present disclosure provides improved syringe filling systems and methods. In one example embodiment, a syringe filling system includes a syringe, a powder source, a funnel, a rotatable auger, and a motor. The syringe has a dispensing end and a barrel chamber. The funnel is configured to hold powder from the powder source. The rotatable auger is positioned within the funnel and is configured to transport powder from the funnel through the dispensing end of the syringe to fill the barrel chamber of the syringe with a predetermined quantity of powder. Additionally, the motor is configured to rotate the auger to cause powder to be transported from the funnel to the syringe.

In another example embodiment, a syringe filling system includes a syringe, a powder source, a funnel, a rotatable auger, and a motor. The syringe has a dispensing end and a barrel chamber. The funnel has an inlet end and an outlet end. Additionally, the funnel has an inside diameter and the funnel is configured to hold powder from the powder source. The rotatable auger is positioned within the funnel, and the auger has a conveying portion with a first diameter, a transition portion, and a filling portion with a second diameter. The auger is configured to transport powder from the funnel through the dispensing end of the syringe to fill the barrel chamber of the syringe with a predetermined quantity of powder. The motor is configured to rotate the auger to cause powder to be transported from the funnel to the syringe.

In another example embodiment, a method for filling a syringe includes positioning a rotatable auger within a funnel. The rotatable auger has a filling portion. Powder is added from a powder source to the funnel, and the outlet end of the funnel is coupled to a dispensing end of the syringe. Additionally, at least a portion of a filling portion of the filling portion of the auger is positioned within the dispensing end of the syringe. The rotatable auger is rotated to convey powder from the funnel to the syringe.

In another example embodiment, a container filling system includes a container, a powder source, a funnel configured to hold powder from the powder source, a rotatable auger, and a motor. The rotatable auger is positioned within the funnel, and the auger is configured to transport powder from the funnel to the container to fill the container with a predetermined quantity of powder. The motor is configured to rotate the auger, and the rotation of the auger causes powder to be transported from the funnel to the container.

It is accordingly an advantage of the present disclosure to improve the quality of powder filled syringes by reducing caking and clogging of the bulk material.

It is another advantage of the present disclosure to reduce the amount of time needed to process and produce luer tip filled syringes.

It is a further advantage of the present disclosure to improve the manufacturing process by reducing syringe contamination.

Additional features and advantages of the disclosed syringe filling systems and methods are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein. Moreover, it should be noted that the language used in the specification has been principally selected for readability and instructional purposes, and not to limit the scope of the inventive subject matter.

### BRIEF DESCRIPTION OF THE FIGURES

FIG. 1 is a schematic view of a syringe filling system according to an example embodiment of the present disclosure.

FIG. 2A is a front view of a funnel according to an example embodiment of the present disclosure.

FIG. 2B is a top view of the funnel of FIG. 2A, according to an example embodiment of the present disclosure.

FIG. 2C is a cross-sectional view of the funnel of FIG. 2A, according to an example embodiment of the present disclosure.

FIG. 3A is a front view of an auger according to an example embodiment of the present disclosure.

FIG. 3B is a top view of the auger of FIG. 3A, according to an example embodiment of the present disclosure.

FIG. 3C is a cross-sectional view of the auger of FIG. 3A, according to an example embodiment of the present disclosure.

FIG. 4 illustrates a flowchart of an example process for filling a syringe, according to an example embodiment of the present disclosure.

### DETAILED DESCRIPTION OF EXAMPLE EMBODIMENTS

As discussed above, syringe filling systems and methods are provided to improve the manufacturing process, quality of the finished filled syringe product, and processing speed. Empty containers (e.g., syringes) are moved down a production line to final packing. A filling station is configured such that the containers are filled through their dispensing ends with powder. A funnel and rotatable auger convey powder from a powder source to the container. For example, the rotatable auger may convey power through the dispensing end of the syringe to fill the syringe barrel.

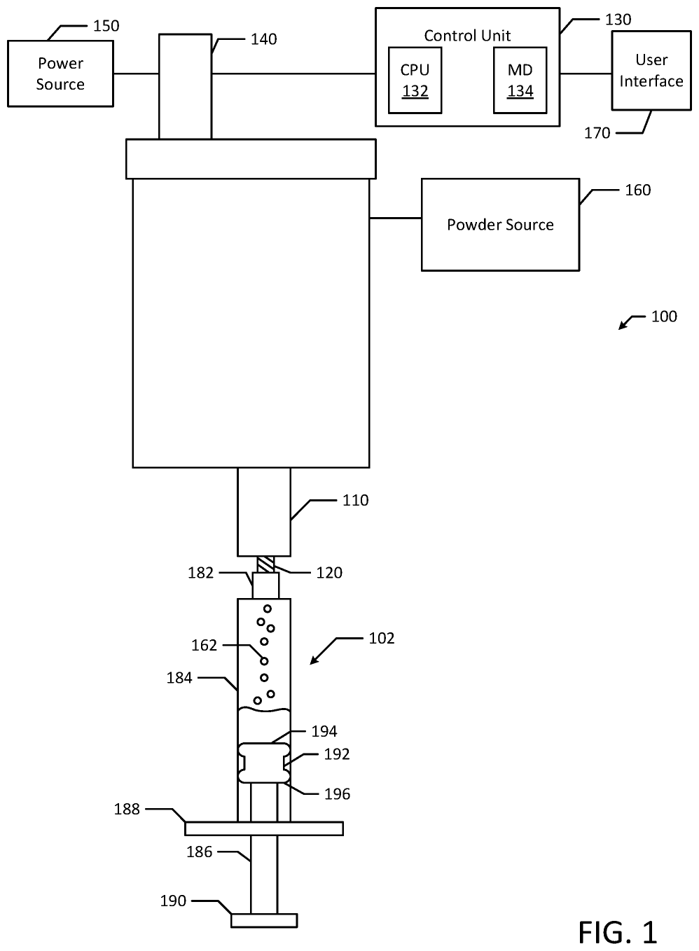

Referring to the drawings and in particular to FIG. 1, in one embodiment, a syringe filling system 100 of the present disclosure is provided to fill containers, such as syringes 102. In one embodiment, the syringe filling system 100 includes a syringe 102, a funnel 110, an auger 120, a control unit 130, a motor 140, a power source 150, and a powder source 160. Control unit 130 may include one or more processor (e.g., CPU 132) and one or more memory (e.g., memory device 134).

As used herein, the term, “processor” may refer to a device capable of executing instructions encoding arithmetic, logical, and/or I/O operations. As used herein, the term, “memory” may refer to a volatile or non-volatile memory device, such as RAM, ROM, EEPROM, or any other device capable of storing data.

Control unit 130 may be programmed to store multiple selectable programs to cause the syringe 102 to be transported along a production line to different filling systems 100. For example, a filling system 100 may be configured for a specific powder 152 or medication and/or a specific syringe size. Additionally, selectable programs may include various predetermined quantities of powder 162 to fill syringes 102. For example, the control unit 130 may be programmed to store multiple programs based on syringe size, powder sources 160, etc.

As illustrated in FIG. 1, syringe 102 includes a dispensing end 182, such as a luer tip. Additionally, syringe 102 includes a barrel chamber 182 and a seal 192 with a leading ring 194 and a trailing ring 196. Syringe 102 also includes a plunger 186, barrel flange 188, and plunger flange 190. The syringe filling system 100 is configured to fill the barrel chamber 182 with a predetermined quantity of powder 162 from powder source 160.

Specifically, auger 120 is configured to convey powder 162 from funnel 110 through the dispensing end 182 and into barrel chamber 184 of syringe 102. For example, auger 120 engages powder in funnel 110 to convey or transport powder towards syringe 102. The powder 162 is carried by the auger through the dispensing end 182 of the syringe and falls into barrel chamber 184. Motor 140 continues to drive (e.g., rotate) auger 120 until the predetermined quantity exits funnel 110 and fills barrel chamber 182 of syringe 102. Filling through the syringe's dispensing end 182 may advantageously eliminate caking of the powder 162. For example, if filled through the barrel end of syringe 102, the powder 162 may cake on lubricating material applied to seal 192. As the plunger 186 and seal 192 are removed from the barrel chamber 182, residue may be left on the inner barrel chamber wall, and powder 162 may stick and/or cake to the wall of barrel chamber 182.

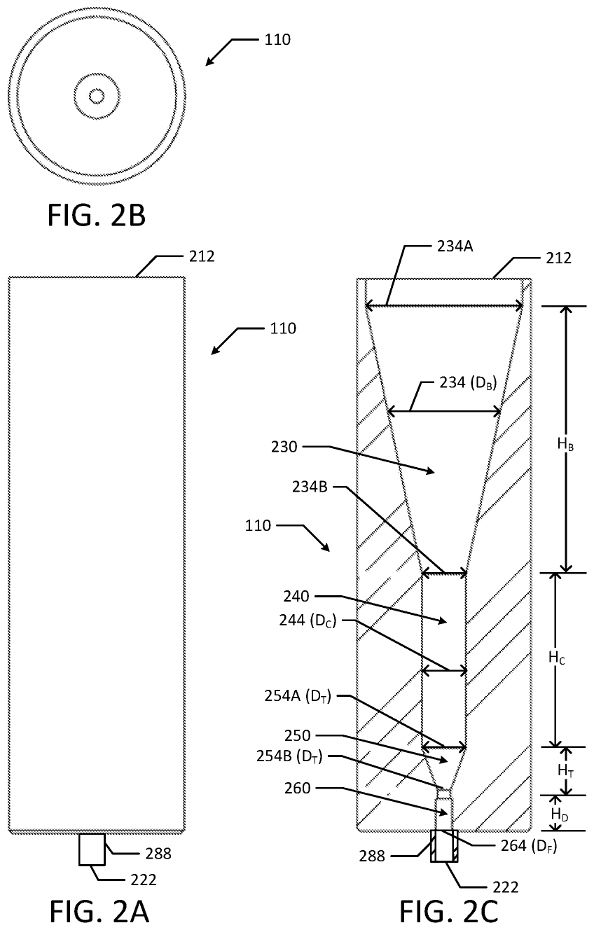

In another example, syringe filling system 100 may be used to fill a barrel end of syringe 102. For example, with plunger 186 removed, the syringe filling system 100 may fill the barrel chamber 182 through the barrel end of syringe 102 with a predetermined quantity of powder 162 from powder source 160. Additionally, filling system 100 may be used to fill other containers, for example, vials. Filling system 100 is adapted to provide precise and accurate filling for a wide variety of powder containers. For example, as discussed in more detail below, delivery tube 288 (as illustrated in FIG. 2A and FIG. 2C), may extend into barrel chamber 184 far enough to avoid any residual residue or lubrication material left on the inner wall of barrel chamber 182 when removing the plunger 186 and seal 192.

Referring now to FIGS. 2A, 2B, and 2C, an example funnel 110 is illustrated. Funnel 110 includes an inlet end 212 and an outlet end 222. In an example, funnel 110 includes a bulk section 230, a conveying section 240, a transition section 250, and a delivery section 260. In the illustrated example, bulk section 230 is adjacent inlet end 212 and in fluid communication with conveying section 240. Conveying section 240 is in fluid communication with delivery section 260 via transition section 250. Delivery section 260 is adjacent outlet end 222. Additionally Funnel 110 may include a delivery tube 288. In an example, delivery tube may be coupled to funnel 110. In another example, delivery tube 288 may be integrated into funnel 110. Delivery tube 288 may be sized and arranged such that it can be inserted into the dispensing end of a syringe 102. Additionally, the delivery tube 288 may extend beyond the end of auger 120 (discussed in more detail below) to provide a barrier between the external environment and the powder conveyed to the syringe. In an example, funnel 110 may be made from stainless steel.

In an example embodiment, bulk section 230 may have an inside diameter (D_{B}) 234 that changes as it moves from a first end to a second end. For example, diameter 234 may linearly decrease from an initial inside diameter 243A at its first end (e.g., the inlet end 212 of funnel 110) to a final inside diameter 234B at its second end. The inside diameter 234 may decrease from approximately 0.90 inches to 0.25 inches. In an example embodiment, the initial inside diameter 234A at the first end may be 0.9005 inches with a tolerance of 0.0005 inches and the final inside diameter 234B at the second end may be 0.252 inches with a tolerance of 0.001 inches.

In an example embodiment, conveying section 240 may have a constant inside diameter (D_{C}) 244. For example, inside diameter 244 of the conveying section 240 may be substantially the same as the final inside diameter 234B of the bulk section 230. For example, inside diameter 244 may be approximately 0.25 inches. In an example embodiment, the inside diameter 244 may be 0.252 inches with a tolerance of 0.001 inches. Transition section 250 may have an inside diameter (D_{T}) 254 that changes. For example, diameter 254 may linearly decrease from an initial inside diameter 254A at its first end (e.g., end adjacent to conveying section 240) to a final inside diameter 254B at its second end (e.g., end adjacent to delivery section 250). The inside diameter 254 may decrease from approximately 0.25 inches to 0.08 inches. In an example embodiment, the initial inside diameter 254A may be 0.252 inches with a tolerance of 0.001 inches and the final inside diameter 254B may be 0.077 inches with a tolerance of 0.001 inches. In an example embodiment, delivery section 260 may have a constant inside diameter (D_{F}) 264. For example, inside diameter 264 of the delivery section 260 may be substantially the same as the final inside diameter 254B of the transition section 250. For example, inside diameter 264 may be approximately 0.08 inches. In an example embodiment, the inside diameter 264 may be 0.077 inches with a tolerance of 0.001 inches.

In an example embodiment, funnel 110 may have an overall height of approximately 3.175 inches. In the illustrated example, bulk section 230 has a height (H_{B}) of approximately 1.542 inches, conveying section 240 has a height (HO of approximately 1.0 inches, transition section 250 has a height (H_{T}) of approximately 0.24 inches, and delivery section 260 has a height (H_{D}) of approximately 0.188 inches.

It should be appreciated that the sizes and dimensions discussed herein are merely for explanation. In various alternative embodiments, the sizing (e.g., diameters, lengths, and heights) of the auger 120 and funnel 110 is different. It should be appreciated that the relative dimensions of the constituent features discussed herein may be ratios rather than strict unitary dimensions. For example, the relationship between bulk section 230 inside diameter (D_{B}) 234, conveying section 240 inside diameter (D_{C}) 244, transition section 250 inside diameter (D_{T}) 254, and delivery section 260 inside diameter (D_{F}) 264 may be described in terms of a ratio(s). Additionally, the varying diameters (D_{B}) 234 and (D_{T}) 254 may be described as a slope or as a percent diameter reduction per unit length. The diameters may also be related to the heights of the funnel 110. Similarly, the relationship between the various funnel 110 section heights may be described in terms of one or more ratios. For example, bulk section 230 height (H_{B}), conveying section 240 height (H_{C}), transition section 250 height (H_{T}), and delivery section 260 height (H_{D}) may be described in terms of a ratio(s). Additionally, the sizes and dimensions discussed herein may be based on syringe size, syringe application, syringe dispensing tip geometry (e.g., luer tip geometry), powder properties (e.g., particle quantity, particle size, bulk particle viscosity), and manufacturing parameters, including but not limited to, rotational speed of auger 120, speed of processing line or fill line, etc. Accordingly, though not all calculated and discussed in detail herein, it should be appreciated that the ratios of respective example dimensions discussed above can be calculated and scaled for alternative goals and embodiments.

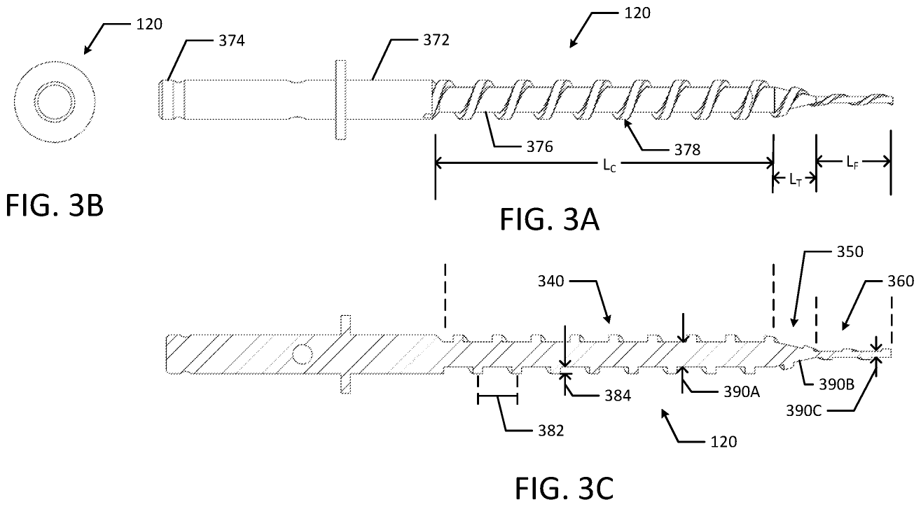

Referring now to FIGS. 3A, 3B, and 3C, an example auger 120 is illustrated. Auger 120 includes a shaft 376 and threads or flight 378. Auger 120 may also include a shank 372 and a tang 374. In the illustrated example, the threaded portion of auger 120 includes a conveying portion 340, a transition portion 350, and a filling portion 360. In the illustrated example, transition portion 350 is positioned between the conveying portion 340 and filling portion 360. During filling operations, filling portion 360 may partially extend into delivery tube 288. For example, during filling operations, both a portion of the delivery tube 288 and the filling portion 360 of auger 120 may be inside dispensing end 182 of syringe 102. In an example, auger 120 may be made from stainless steel.

Threads or flight 378 have pitch 382 and a thread height 384. In an example embodiment, the pitch 382 may be 0.25 inches and thread height 384 may be approximately 0.045 inches in the conveying portion 340. At the filling portion 360, thread height 384 may be reduced to 0.012 inches. In an example, the thread height may be adjusted to change the quantity or amount of powder that the auger conveys per rotation. For example, taller threads (e.g., larger thread heights) may convey larger quantities of powder 162 per rotation since the additional height supports or carries more powder (e.g., there is more thread or flight surface to support the powder). Additionally, smaller thread heights may convey less powder 162 per auger 120 rotation because there is less thread surface to support the powder.

Conveying portion 340 may have a constant shaft diameter 390A. For example, shaft diameter 390A may be approximately 0.160 inches. Shaft diameter 390B of the transition portion 350 may change as it transitions from conveying portion 340 to filling portion 360. Filling portion 360 may have a constant shaft diameter 390C. For example, shaft diameter 390C may be approximately 0.040 inches. For example, inside diameter 264 may be approximately 0.08 inches. In an example embodiment, the inside diameter 264 may be 0.077 inches with a tolerance of 0.001 inches.

The threaded portion of auger 120 may have an overall length of approximately 3.0 inches. In the illustrated example, conveying portion 340 has a length (L_{C}) of approximately 2.173 inches, transition portion 350 has a length (L_{T}) of approximately 0.262 inches, and filling portion 360 has a length (L_{F}) of approximately 0.490 inches.

As discussed above, thread height 384, pitch 382, shaft diameter 290, screw diameter (e.g., diameter of shaft accounting thread height), shaft length, conveying portion 340 length (L_{C}), transition portion 350 length (L_{T}), and filling portion 360 length (L_{F}) can vary based on one or more of the complimentary funnel 110, filling apparatus, and syringe 102 appropriate for perspective application of the filling device or system 100. Similarly, as discussed above, the relative dimensions of the auger 120 features may be ratios rather than strict unitary dimensions.

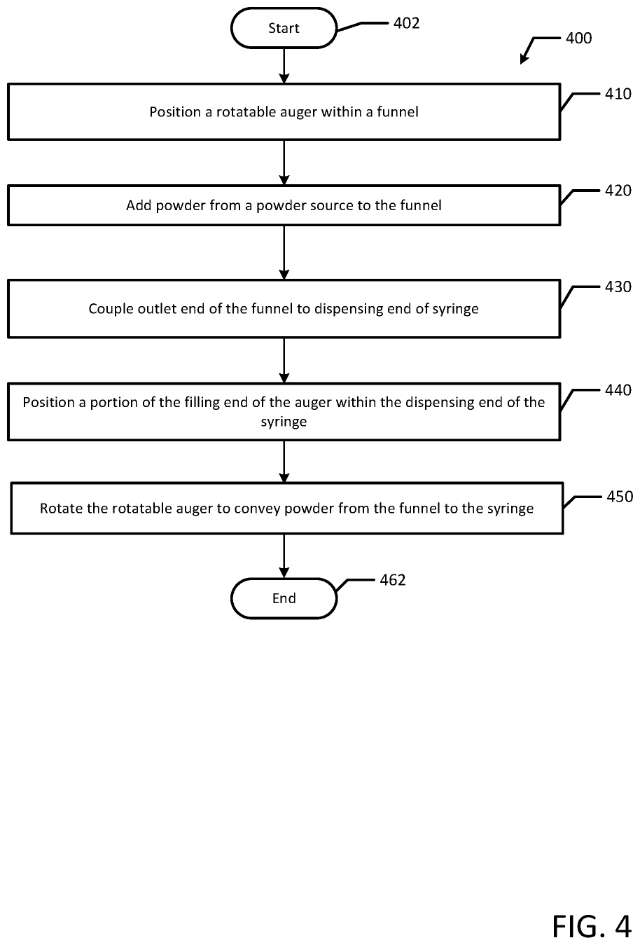

FIG. 4 illustrates a flowchart of an example method 400 for filling a syringe in accordance with an example embodiment of the present disclosure. The flowchart of FIG. 4 may be implemented at control unit 130. Although the example method 400 is described with reference to the flowchart illustrated in FIG. 4, it should be appreciated that many other methods of performing the acts associated with the method 400 may be used. For example, the order of some of the blocks may be changed, certain blocks may be combined with other blocks, and some of the blocks described are optional. The method 400 may be performed by processing logic that may include hardware (circuitry, dedicated logic, etc.), software, or a combination of both.

At oval 402, method 400 begins. At block 410, example method 400 positions a rotatable auger 120 within a funnel 110. For example, rotatable auger 120 is positioned such that the filling portion 360 partially extends beyond the outlet end 222 of funnel 110. At block 420, example method 400 adds powder 162 from a powder source 160 to the funnel 110. At block 430, example method 400 couples the outlet end 222 of the funnel 110 to a dispensing end 182 of the syringe 102. For example, funnel 110 may be fitted with a luer lock connection that engages a luer tip of the syringe 102. At block 440, example method 400 positions a portion of the filling portion 360 of the auger 120 within the dispensing end 182 the syringe 102. For example, as the outlet end 222 of the funnel 110 is coupled to the syringe 102, the filling portion 360 of the auger extends into the dispensing end 182 of the syringe 102. At block 450, example method 400 also rotates the rotatable auger 120 to convey powder 162 from the funnel 110 to the syringe 102. As the auger 120 rotates, powder 162 is carried along threads or flight 378 from the conveying portion 340 of the auger 120 towards to filling portion 360. The auger 120 and funnel 110 are configured and arranged such that the powder 162 in contact with auger 120 is conveyed downward towards syringe 102. As the auger rotates, powder traveling along the threads or flight 378 falls off the end of the filling portion 360 and into the syringe 102. At oval 462, method 400 ends. Powder 162 is prevented from exiting funnel 110 when the auger 120 is stationary from the viscosity of the powder and the interactions between the powder 162, auger 120, and/or funnel 110. For example, the powder 162 may have sufficient viscosity such that the powder 162 does not exit funnel 110. Additionally, friction between particles of powder 162, auger 120, and/or funnel 110 may prevent the powder 162 from existing funnel 110. Other interactions such as mechanical interlocking and inter-particulate cohesion may also prevent powder 162 from exiting funnel 110 when auger 120 is stationary.

In another example, method 400 may be used to fill a syringe 102 through its barrel end, which may advantageously eliminate caking of the powder 162. Additionally, method 400 may be used to fill other containers, such as a medical vial. For example, method 400 may be used to provide precise and accurate filling for a wide variety of powder containers.

Aspects of the subject matter described herein may be useful alone or in combination with one or more other aspects described herein. In an exemplary aspect of the present disclosure, a syringe filling system includes a syringe having a dispensing end and a barrel chamber, a powder source, a funnel configured to hold powder from the powder source, a rotatable auger positioned within the funnel, and a motor. The auger is configured to transport powder from the funnel through the dispensing end of the syringe to fill the barrel chamber of the syringe with a predetermined quantity of powder. The motor is configured to rotate the auger. Additionally rotation of the auger causes powder to be transported from the funnel to the syringe.

In another exemplary aspect of the present disclosure, which may be used in combination with any one or more of the preceding aspects, the dispensing end includes a luer tip.

In another exemplary aspect of the present disclosure, which may be used in combination with any one or more of the preceding aspects, the powder is configured to seal a wound.

In another exemplary aspect of the present disclosure, which may be used in combination with any one or more of the preceding aspects, the powder is a hemostatic powder.

In another exemplary aspect of the present disclosure, which may be used in combination with any one or more of the preceding aspects, the funnel has an outlet end having an inside diameter, the auger having a filling portion with an outside diameter, the outside diameter being smaller than the inside diameter.

In another exemplary aspect of the present disclosure, which may be used in combination with any one or more of the preceding aspects, the funnel is configured to present powder to the auger.

In another exemplary aspect of the present disclosure, which may be used in combination with any one or more of the preceding aspects, the auger has a conveying portion, a transition portion, and a filling portion.

In another exemplary aspect of the present disclosure, which may be used in combination with any one or more of the preceding aspects, the conveying portion has a first diameter and the filling portion has a second diameter, the second diameter small than the first diameter.

In another exemplary aspect of the present disclosure, which may be used in combination with any one or more of the preceding aspects, the transition portion has a first end having the first diameter and a second end having the second diameter.

In another exemplary aspect of the present disclosure, which may be used in combination with any one or more of the preceding aspects, the transition portion has a first end having the first diameter and a second end having the second diameter.

Aspects of the subject matter described herein may be useful alone or in combination with one or more other aspects described herein. In an exemplary aspect of the present disclosure, a syringe filling system includes a syringe having a dispensing end and a barrel chamber, a powder source, a funnel, a rotatable auger, and a motor. The funnel has an inlet end and an outlet end. Additionally, the funnel has an inside diameter and the funnel is configured to hold powder from the powder source. The rotatable auger is positioned within the funnel. The auger has a conveying portion with a first diameter, a transition portion, and a filling portion with a second diameter. The auger is configured to transport powder from the funnel through the dispensing end of the syringe to fill the barrel chamber of the syringe with a predetermined quantity of powder. The motor is configured to rotate the auger, where rotation of the auger causes powder to be transported from the funnel to the syringe.

In another exemplary aspect of the present disclosure, which may be used in combination with any one or more of the preceding aspects, the dispensing end includes a luer tip.

In another exemplary aspect of the present disclosure, which may be used in combination with any one or more of the preceding aspects, the powder is configured to seal a wound.

In another exemplary aspect of the present disclosure, which may be used in combination with any one or more of the preceding aspects, the powder is a hemostatic powder.

Aspects of the subject matter described herein may be useful alone or in combination with one or more other aspects described herein. In an exemplary aspect of the present disclosure, a method for filling a syringe includes positioning a rotatable auger within a funnel, the rotatable auger having a filling portion, adding powder from a powder source to the funnel, coupling the outlet end of the funnel to a dispensing end of the syringe, positioning at least a portion of the filling portion of the auger within the dispensing end of the syringe, and rotating the rotatable auger to convey powder from the funnel to the syringe.

In another exemplary aspect of the present disclosure, which may be used in combination with any one or more of the preceding aspects, the method further includes ceasing rotation of the rotatable auger once a predetermined quantity of the powder is conveyed to the syringe.

Aspects of the subject matter described herein may be useful alone or in combination with one or more other aspects described herein. In an exemplary aspect of the present disclosure, a container filling system includes a container, a powder source, a funnel configured to hold powder from the powder source, a rotatable auger positioned within the funnel, and a motor. The auger is configured to transport powder from the funnel to the container to fill the container with a predetermined quantity of powder. The motor is configured to rotate the auger. Additionally, rotation of the auger causes powder to be transported from the funnel to the container.

In another exemplary aspect of the present disclosure, which may be used in combination with any one or more of the preceding aspects, the container is one of a syringe and a vial.

In another exemplary aspect of the present disclosure, which may be used in combination with any one or more of the preceding aspects, the container is a syringe and the syringe has a dispensing end and a barrel end.

In another exemplary aspect of the present disclosure, which may be used in combination with any one or more of the preceding aspects, the rotation of the auger causes powder to be transported from the funnel to the syringe through the barrel end of the syringe.

To the extent that any of these aspects are mutually exclusive, it should be understood that such mutual exclusivity shall not limit in any way the combination of such aspects with any other aspect whether or not such aspect is explicitly recited. Any of these aspects may be claimed, without limitation, as a system, method, apparatus, device, medium, etc.

The many features and advantages of the present disclosure are apparent from the written description, and thus, the appended claims are intended to cover all such features and advantages of the disclosure. Further, since numerous modifications and changes will readily occur to those skilled in the art, the present disclosure is not limited to the exact construction and operation as illustrated and described. Therefore, the described embodiments should be taken as illustrative and not restrictive, and the disclosure should not be limited to the details given herein but should be defined by the following claims and their full scope of equivalents, whether foreseeable or unforeseeable now or in the future.

1. A syringe filling system comprising:
a syringe having a dispensing end and a barrel chamber;
a powder source;
a funnel configured to hold powder from the powder source;
a rotatable auger positioned within the funnel, wherein the auger is configured to transport powder from the funnel through the dispensing end of the syringe to fill the barrel chamber of the syringe with a predetermined quantity of powder; and
a motor configured to rotate the auger, wherein rotation of the auger causes powder to be transported from the funnel to the syringe.

2. The system of claim 1, wherein the dispensing end includes a luer tip.

3. The system of claim 1, wherein the powder is configured to seal a wound.

4. The system of claim 1, wherein the powder is a hemostatic powder.

5. The system of claim 1, wherein the funnel has an outlet end having an inside diameter, the auger having a filling portion with an outside diameter, the outside diameter being smaller than the inside diameter.

6. The system of claim 1, wherein the funnel is configured to present powder to the auger.

7. The system of claim 1, wherein the auger has a conveying portion, a transition portion, and a filling portion.

8. The system of claim 7, wherein the conveying portion has a first diameter and the filling portion has a second diameter, the second diameter small than the first diameter.

9. The system of claim 8, wherein the transition portion has a first end having the first diameter and a second end having the second diameter.

10. The system of claim 1, wherein the funnel includes an inlet end, an outlet end, a bulk section adjacent the inlet end, a conveying section, a transition section, and a delivery section adjacent the outlet end, and wherein the conveying section is in fluid communication with the delivery section via the transition section.

11. A syringe filling system comprising:
a syringe having a dispensing end and a barrel chamber;
a powder source;
a funnel with an inlet end and an outlet end, the funnel having an inside diameter, the funnel configured to hold powder from the powder source;
a rotatable auger positioned within the funnel, the auger having a conveying portion with a first diameter, a transition portion, and a filling portion with a second diameter, wherein the auger is configured to transport powder from the funnel through the dispensing end of the syringe to fill the barrel chamber of the syringe with a predetermined quantity of powder; and
a motor configured to rotate the auger, wherein rotation of the auger causes powder to be transported from the funnel to the syringe.

12. The system of claim 11, wherein the dispensing end includes a luer tip.

13. The system of claim 11, wherein the powder is configured to seal a wound.

14. The system of claim 11, wherein the powder is a hemostatic powder.

15. A method for filling a syringe comprising:
positioning a rotatable auger within a funnel, the rotatable auger having a filling portion;
adding powder from a powder source to the funnel;
coupling the outlet end of the funnel to a dispensing end of the syringe;
positioning at least a portion of the filling portion of the auger within the dispensing end of the syringe; and
rotating the rotatable auger to convey powder from the funnel to the syringe.

16. The method of claim 15, further comprising ceasing rotation of the rotatable auger once a predetermined quantity of the powder is conveyed to the syringe.

17. A container filling system comprising:
a container;
a powder source;
a funnel configured to hold powder from the powder source;
a rotatable auger positioned within the funnel, wherein the auger is configured to transport powder from the funnel to the container to fill the container with a predetermined quantity of powder; and
a motor configured to rotate the auger, wherein rotation of the auger causes powder to be transported from the funnel to the container.

18. The system of claim 17, wherein the container is one of a syringe and a vial.

19. The system of claim 17, wherein the container is a syringe, the syringe having a dispensing end and a barrel end.

20. The system of claim 19, wherein the rotation of the auger causes powder to be transported from the funnel to the syringe through the barrel end of the syringe.
